(19) United States
(12) Patent Application Publication
(10) Pub. No.: US 2004/0014122 A1
(43) Pub. Date: Jan. 22, 2004

(54) Detection of spore forming bacteria

(76) Inventors: Alexander W. Breen, Jacksonville, FL (US); Freddie L. Singleton, Switzerland, FL (US)

Correspondence Address: Joanne Mary Fobare Rossi, Hercules Incorporated, Hercules Plaza, 1313 North Market Street, Wilmington, DE, 19894-0001, US

(21) Appl. No.: 10/608,062

(22) Filed: Jun. 27, 2003

Related U.S. Application Data
(63) Continuation of application No. 09/492,135, filed on Jan. 27, 2000, now abandoned.
Application No. 09/492,135 is a continuation-in-part of application No. 09/356,677, filed on Jul. 20, 1999, now abandoned.
Application No. 09/356,677 is a continuation of application No. 09/085,359, filed on May 27, 1998, now Pat. No. 5,928,875.

(51) Int. Cl. C12Q 1/68; C07H 21/04

(52) U.S. Cl. 435/6; 536/24.3

(57) ABSTRACT

Methods for the systematic identification of sporulation genes in spore forming bacteria comprising amplifying a portion of a gene from total cellular DNA of said spore forming bacteria by using a primer; and detecting the presence of the amplification product.

### RELATED APPLICATIONS

The present application is a continuation application of U.S. application Ser No. 09/492,135 filed Jan. 27, 2000, which is a continuation-in-part application of U.S. application Ser. No. 09/356,677 filed Jul. 19, 1999, which is a continuation application of U.S. application Ser. No. 09/085,359 filed May 27, 1998, which has issued as U.S. Pat. No. 5,928,875. The entire disclosure of application Ser. Nos. 09/492,135 and 09/356,677 are considered as being part of the disclosure of this application, and the entire disclosure of application Ser. Nos. 09/356,677 and 09/492,135 are expressly incorporated by reference herein in their entireties.

### BACKGROUND OF THE INVENTION

1. Field of the Invention

This invention relates to methods for the detection of spore forming bacteria. The invention is particularly useful in detecting bacteria in paper products and paper manufacturing streams. Detection of spore forming bacteria according to the present invention includes methods involving polymerase chain reaction. Primers particularly suitable for use in detection of spore forming bacteria are disclosed as well.

2. Background of the Invention and Related Information

Paper products used in the packaging of food should be free from the presence of microorganisms which adversely affect the hygiene of the food. The most common route of contamination by these microorganisms is during the manufacturing of the paper products, where the microorganisms can grow and flourish. Commonly, such contamination is dealt with through the use of biocides or heat. However, biocide usage may be limited by risks at both the paper mill, and in the final paper product. Additionally, some microorganisms are able to avoid eradication by their inherent protection mechanism—sporulation.

In the paper industry, one of the more costly and persistent problems is control of spore forming bacteria (SFB). Unlike most bacteria, SFB can pass through dryer sections of a mill to pose a contamination threat when the paper product is used, for example, in food packaging. Also, spore forming bacteria are frequently resistant to all but the most toxic of biocides. A number of SFB have been identified as problematic in papermaking, and have been described by Pirttijärvi and others in Journal of Applied Bacteriology 81, 445-458 (1996), the entire contents of which are hereby incorporated by reference.

A number of industry trends have generated even more concern over the microbiological quality of paper used for food packaging. Recycled fiber which often contains starch and coating material can support microbial growth. As the fraction of recycled material going into production increases, so will the chance for contamination of the finished product. Coinciding with this increase in recycled fiber is a desire to decrease the use of biocides for control of microbial growth. Fast, reliable, simple and cost-effective monitoring of product quality will increase overall production efficiency by allowing problematic populations to be controlled while at the same time permitting biocides to be applied when, and at the specific location needed.

The current industry standard for food packaging grade material in the United States is 250 spores per gram of paper. This is determined by the Dairyman's method, a plate count enumeration technique which requires a 48-hour incubation period. A more rapid diagnosis of a contamination problem would result in significantly less wasted product and an overall increase in mill productivity.

The need to rapidly detect spore forming bacteria is not limited to paper making processes. For example, the heat resistant spores formed by members of Bacillus, Paenbacillus, and Clostridium, for example, can be problematic in food, pharmaceutical, and medical product processing, where heat sterilization under pressure is not appropriate. In these processes, special care must be taken to avoid contamination and to evaluate sources of contamination when present. A rapid identification of a contaminating source material can often prevent unnecessary production stoppages, and may save thousands of dollars.

The need to identify spore forming bacteria also arises in medical treatment. Occasionally, for example, in the treatment of a bacterial infection, e.g., bronchitis, upper respiratory tract infection, earache, etc., the antibiotic selected is effective against the organism causing the infection but fails to kill a population of bacteria such as a Clostridium strain (a spore forming bacteria). While the Clostridium is normally not problematic, in the absence of competition from other organisms (which are killed by the original course of antibiotics), the Clostridium thrives, causing a potentially serious infection. Thus, there is a need for detecting the presence of such species in a biological sample.

### SUMMARY OF THE INVENTION

The present invention is directed to methods for detecting the presence of bacteria. More particularly, the present invention is directed to methods for detecting the presence of bacteria using nucleotide primers. In particular, the present invention is directed to detecting spore forming bacteria with such primers. Detection methods according to the present invention include the use of polymerase chain reaction in conjunction with electrophoresis, or fluorescence techniques. The present invention is further directed to nucleotide primers, and more particularly, to sets of nucleotide primers, which are used in the detection of spore forming bacteria.

These and other aspects of the present invention are achieved by the provision of methods for the systematic identification of sporulation genes in spore forming bacteria comprising amplifying a portion of a gene from total cellular DNA of the spore forming bacteria by using a primer group comprising 5′-AGTATCATTCATGAAATTGG-3′ (SEQ ID NO. 1), 5′-AAAAAAGCAGTTGACT-3′ (SEQ ID NO. 2), 5′-CGGCTTGCCGTTGTATT-3′ (SEQ ID NO. 3), 5′-GAAGATGTGACGAAAAAG-3′ (SEQ ID NO. 4), 5′-CAAGAAGATGTGACGAAA-3′ (SEQ ID NO. 5), 5′-GTTGTATTATATTTCTTTGC-3′ (SEQ ID NO. 6), and 5′-GTTGTGTTAAATTTTTTGGC-3′ (SEQ ID NO. 7), and 5′-AGTATCATTCATGAAATTGGCGTTCC-3′ (SEQ ID NO. 8); and detecting the presence of the amplification product. Spore forming bacteria include, but are not limited to, Bacillus megaterium, Bacillus lichenformis, Bacillus cereus group, Bacillus pumilus, as well as Paenbacillus macerans, Paenbacillus polymyxa, Paenbacillus pabuli, Bacillus flexus, Bacillus subtilis, Bacillus anthracis, Bacillus sporothermodurans, Bacillus sphaericus, Clostridium perfringens, Clostridium butyricum, Clostridium pasteurianum, Clostridium cochlearium, Clostridium scatologenes, Clostridium sordellii, Clostridium lituseburense, Clostridium paradoxum, Clostridium thermocellum, Thermoanaerobacter brockii, Moorella thermoautotrophica, Sporomusa ovata, Thermobrachium celere, Bacillus acidocaldarus, Bacillus amyloliquefaciens, Bacillus brevis, Bacillus thuringiensis, Bacillus stearothermophilus, Clostridium dificile, Clostridium cellulolyticum, Clostridium bifermentans, and Clostridium acetoburylicum. Amplifying may include the use of polymerase chain reaction, and detecting may include electrophoresing the amplification product and visualizing an electrophoresis substrate with staining. In some embodiments, the electrophoresis substrate comprises agarose gel; in some embodiments, staining comprises applying ethidium bromide.

Another aspect of the present invention includes a primer pair comprising a member selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, and SEQ ID NO. 8, and another aspect of the present invention includes a nucleotide sequence, which may be a primer or probe, comprising a sequence selected from the group consisting of SEQ ID NO. 1, SEQ ID NO. 2, SEQ ID NO. 3, SEQ ID NO. 4, SEQ ID NO. 5, SEQ ID NO. 6, SEQ ID NO.7, and SEQ ID NO. 8. The present invention also includes primers selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, and SEQ ID NO. 8.

The present invention is still further directed to a composition comprising at least one cellulose-containing material and at least one primer comprising a sequence selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, and SEQ ID NO. 8. The cellulose-containing material may comprise paper pulp.

Also within the scope of the present invention are kits for testing for the presence of spore forming bacteria, wherein the kits comprise at least one primer comprising a sequence selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, and SEQ ID NO. 8, and at least one media supportive of spore forming bacterial growth. The kits may be designed for use in testing paper-making processes, or may be generic to testing any material. Methods of using such kits are also within the scope of the present invention.

The present invention is still further directed to methods for testing a sample for the presence of spore forming bacteria, wherein the methods comprise a) combining at least two nucleic acid primers complimentary to at least one forward and at least one reverse nucleic acid sequence from the total cellular DNA of the bacteria, with a sample; amplifying cellular DNA of bacteria in the sample with primers; and c) detecting presence of amplified DNA. The sample may be a cellulose-containing sample and may be a sample taken from a paper making process. Such samples include, but are not limited to, samples from white water, head box, broke, additive storage tank, and coated calender. Other samples include air, soil, water, blood, fecal matter, starch, protein, or an epichlorohydrin reaction product. Any of the nucleotide sequences disclosed in the present application may be used for the primer pairs, and such sequences include SEQ ID NO. 1, SEQ ID NO. 2, SEQ ID NO. 3, SEQ ID NO. 4, SEQ ID NO. 5, SEQ ID NO. 6, SEQ ID NO. 7, and SEQ ID NO. 8.

The present invention also provides methods for testing cellulose-containing samples for the presence of spore forming bacteria, wherein the methods comprise combining at least one primer comprising a sequence selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, and SEQ ID NO. 8 with a cellulose-containing sample. The present invention is also directed to methods for testing a cellulose-containing sample for the presence of spore forming bacteria, the methods comprising a) combining at least two nucleic acid primers complimentary to at least one forward and at least one reverse nucleic acid sequence from the total cellular DNA of the bacteria with a cellulose-containing sample; and b) visualizing hybridized primers. The at least two nucleic acid primers preferably comprise at least one of SEQ ID NO. 1, SEQ ID NO. 2, SEQ ID NO. 3, SEQ ID NO. 4, SEQ ID NO. 5, SEQ ID NO. 6, SEQ ID NO. 7, and SEQ ID NO. 8.

The present invention also provides methods for controlling a population of spore forming bacteria in an industrial process stream, the methods comprising a) detecting bacteria in the process stream using a primer comprising a sequence selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, and SEQ ID NO. 8; and b) adjusting a biocide concentration in the process stream sufficient to reduce the number of bacteria. The industrial process stream may be, for example, a paper making process stream, or a food processing stream.

Other aspects of the present invention include methods for the systematic identification of sporulation genes in spore forming bacteria, the methods comprising: a) amplifying a portion of a gene from total cellular DNA of the spore forming bacteria by using at least one of SEQ ID NO. 1, SEQ ID NO. 2, SEQ ID NO. 3, SEQ ID NO. 4, SEQ ID NO. 5, SEQ ID NO. 6, SEQ ID NO. 7, and SEQ ID NO. 8; and b) detecting the presence of an amplification product.

The present invention also provides probes for detecting the presence of spore forming bacteria in a sample, the probe comprising a nucleotide sequence able to form a detectable hybrid with spo0A gene of spore forming bacteria and unable to form a detectable hybrid with genetic material of non-spore forming bacteria, wherein the nucleotide sequence consists essentially of adenine, guanine, cytosine, and thymine. Such nucleotide sequences may comprise SEQ ID NO. 1, SEQ ID NO. 2, SEQ ID NO. 3, SEQ ID NO. 4, SEQ ID NO. 5, SEQ ID NO. 6, SEQ ID NO. 7, or SEQ ID NO. 8.

Another embodiment of the present invention is a probe for detecting the presence of spore forming bacteria in a sample, the probe comprising a nucleotide sequence able to form a detectable hybrid with spo0A gene of spore forming bacteria and unable to form a detectable hybrid with genetic material of non-spore forming bacteria, wherein the nucleotide sequence is able to form a detectable hybrid to bases 76 to 93 of the spo0A gene of Bacillus cereus, corresponding to GenBank accession number gb U09972, SEQ ID NO. 49. This nucleotide sequence may comprise SEQ ID NO. 5.

The present invention also provides a probe for detecting the presence of spore forming bacteria in a sample, the probe comprising a nucleotide sequence able to form a detectable hybrid with spo0A gene of spore forming bacteria and unable to form a detectable hybrid with genetic material of non-spore forming bacteria, wherein the nucleotide sequence is able to form a detectable hybrid to bases 403 to 422 of the spo0A gene of Bacillus cereus, corresponding to GenBank accession number gb U09972, SEQ ID NO. 50. The nucleotide sequence may comprise SEQ ID NO. 6 or SEQ ID NO. 7.

The present invention is also directed to compositions comprising at least one primer comprising a sequence selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, and SEQ ID NO. 8. The composition may also include a cellulose-containing material, such as paper pulp.

Aspects of the present invention include methods for testing samples for the presence of spore forming bacteria, the methods comprising a) combining at least two nucleic acid primers complimentary to at least one forward and at least one reverse nucleic acid sequence from the total cellular DNA of the bacteria with a sample; and b) visualizing hybridized primers; wherein the at least two nucleic acid primers comprise at least one of SEQ ID NO. 1, SEQ ID NO. 2, SEQ ID NO. 3, SEQ ID NO. 4, SEQ ID NO. 5, SEQ ID NO. 6, SEQ ID NO. 7, and SEQ ID NO. 8. Samples which may be tested include, but are not limited to, samples of air, soil, water, blood, fecal matter, starch, protein, and/or an epichlorohydrin reaction product.

Another aspect of the present invention includes probes for detecting the presence of spore forming bacteria in a sample, the probes comprising a nucleotide sequence able to form a detectable hybrid with spo0A gene of spore forming bacteria and unable to form a detectable hybrid with genetic material of non-spore forming bacteria, wherein the nucleotide sequence is able to form a detectable hybrid to bases 70 to 427 of the spo0A gene of Bacillus cereus, SEQ ID NO. 51, the nucleotide sequence consisting essentially of guanine, cytosine, adenine, and thymine.

Another aspect of the present invention includes probes for detecting the presence of spore forming bacteria in a sample, the probes comprising a nucleotide sequence able to form a detectable hybrid with spo0A gene of spore forming bacteria and unable to form a detectable hybrid with genetic material of non-spore forming bacteria, wherein the nucleotide sequence is able to form a detectable hybrid to bases 70 to 427 of the spo0A gene of Bacillus cereus, SEQ ID NO. 51, the nucleotide sequence comprising at least one of SEQ ID NO. 1, SEQ ID NO. 2, SEQ ID NO. 3, SEQ ID NO. 4, SEQ ID NO. 5, SEQ ID NO. 6, SEQ ID NO. 7, and SEQ ID NO. 8.

Another aspect of the present invention includes methods of making nucleotide sequences for detecting the presence of a conserved gene in spore forming bacteria, the methods comprising a) determining conserved regions of the conserved gene from at least two strains of spore forming bacteria; and b) preparing nucleotide sequences able to hybridize to the conserved regions, wherein the nucleotide sequences consist essentially of adenine, guanine, cytosine, and thymine. The conserved gene may comprise spo0A, ssp, and/or dpaA/B, but is preferably spo0A.

The present invention is also directed to systems for identifying spore forming bacteria, the systems comprising: a) means for rendering DNA of the spore forming bacteria susceptible to hybridization with at least one nucleotide primer; b) at least one nucleotide primer; and c) means for detecting the hybridization of the DNA of the spore forming bacteria to the at least one nucleotide primer. The DNA of the spore forming bacteria may comprise the spo0A gene, and the at least one nucleotide primer may consist essentially of adenine, guanine, cytosine, and thymine. The at least one nucleotide primer may comprise a sequence selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, and SEQ ID NO. 8. The means for rendering DNA of the spore forming bacteria susceptible to hybridization may comprise a growth step in which the bacteria are placed in an environment which encourages growth, followed by a lysis step in which the bacteria are lysed. The lysis step may comprise heating. The means for detecting the hybridization may comprise polymerase chain reaction. The means for detecting the hybridization may comprise a fluorescence detection technique.

### DETAILED DESCRIPTION OF THE INVENTION

The present invention is directed to methods for detecting bacteria, and in particular, spore forming bacteria (SFB). Spore forming bacteria are those bacteria which have the ability to form spores, and such bacteria are well known in the art. Examples of such SFB include, but are not limited to, Bacillus megaterium, Bacillus lichenformis, Bacillus cereus group, Bacillus pumilus, as well as Paenbacillus macerans, Paenbacillus polymyxa, Paenbacillus pabuli, Bacillus flexus, Bacillus subtilis, Bacillus anthracis, Bacillus sporothermodurans, Bacillus sphaericus, Clostridium perfringens, Clostridium butyricum, Clostridium pasteurianum, Clostridium cochlearium, Clostridium scatologenes, Clostridium sordellii, Clostridium lituseburense, Clostridium paradoxum, Clostridium thermocellum, Thermoanaerobacter brockii, Moorella thermoautotrophica, Sporomusa ovata, Thermobrachium celere, Bacillus acidocaldarus, Bacillus amyloliquefaciens, Bacillus brevis, Bacillus thuringiensis, Bacillus stearothermophilus, Clostridium dificile, Clostridium cellulolyticum, Clostridium bifermentans, and Clostridium acetoburylicum.

The present invention is useful in the detection of SFB in paper making processes, but is not limited to such processes. (As used herein, the term “paper” is to be used in the generic sense. That is, “paper,” as in a “paper making process,” is meant to include paper, paperboard, cardboard, etc.) When used for testing in paper making processes, the process water itself may be tested. The process water may be tested anywhere in the process, but is preferably tested in head boxes or storage tanks. Such storage tanks may contain paper making additives which are to be tested for the presence of SFB. Such additives include starch, latex, clays, proteins, and epichlorohydrin reaction products, including but not limited to reaction products of poly(adipic acid-co-diethylenetriamine) and epichlorohydrin, sold under the trade name Kimene. In addition to testing process water in the paper making process, the paper making machine may be tested for the presence of SFB. Frequently, it is preferable to test shower head deposits for the presence of SFB.

The present invention may also be used in detecting SFB in air, soil, food, and water, including waste water, industrial process water, and drinking water. The present invention may be used in the detection of SFB in protein-containing samples. The present invention may be used in the detection of SFB in medical diagnostic applications, including, for example, testing for at least one SFB in blood or fecal matter. The methods for detecting bacteria in these other media are similar to those for detection in paper making, as described herein.

The present invention focuses on the evolutionary conservation of genes mediating the process of sporulation. A subset of phylogenetically diverse bacteria are able to form spores. Most commonly found spore forming bacteria are members of the genus Bacillus (aerobic bacteria) and Clostridium (anaerobic bacteria). Sporulation is a complicated developmental process, responsive to adverse environmental conditions and under strict physiological control of the cell. Heat, starvation, and chemical perturbation include some but not all of the factors that may induce the sporulation pathway. Genes involved early in the sporulation process are highly homologous across species boundaries. Spo0A, one such gene, may be considered a “master switch” in the sporulation process.

The spo0A gene encodes a kinase responsible for signaling, via phosphorylation, other genes in the process to become active. The phosphorylation state of the spo0A kinase dictates its activity in the cell. Due to this central role in triggering sporulation, spo0A is a highly conserved gene and hence a good target gene for detection.

The present invention is based on the discovery that spore forming bacteria have some conserved genetic material that may be targeted in their detection. The conserved genetic material targeted in accordance with the present invention is the spo0A gene, or a gene homologous thereto. By targeting this gene (or a homologous gene), the present invention is able to detect a very broad range of bacteria. Each of the bacteria detectable according to the present invention is believed to have the spo0A gene, or a gene homologous thereto, which may be involved in sporulation. Other genes which may be targeted in accordance with the present invention include the ssp gene and the dpaA/B gene, each of which is present in sporogenic bacteria and absent in asporogenic bacteria.

The concept underlying the present invention is the discovery that specific, short chains of nucleotides, can bind to the genetic material of the targeted bacteria. Through a number of different techniques, this binding can be visualized or even quantified. The basic underlying technology of the use of nucleic acid probes, or primers, to identify target genetic material is well known in the art, and has been described elsewhere. For their discussion of spore forming bacteria, and methods for their detection using the spo0A gene, Brill and Wiegel (Journal of Microbiological Methods 31 (1997) 29-36), and Brown et al. (Molecular Microbiology 14(3) (1994) 411-426), are hereby incorporated by reference. For their discussion of the use of probes and primers for identifying bacteria, U.S. Pat. Nos. 5,747,252, 5,969,122, 5,430,137, 5,714,321, and 5,958,679, are hereby incorporated by reference.

Thus, the present invention is directed to the use of nucleotide sequences for targeting specific portions of the spo0A gene. These nucleotide sequences can bind, or hybridize, to target portions of the SFB genetic material. The target portion of the spo0A gene spans bases beginning at about 70 and ending at about 427 of Bacillus cereus, GenBank accession #gb U09972, SEQ ID NO. 51. The nucleotide sequences of the present invention can also target homologous sequences from other SFB.

Obviously, the numbering of the bases will differ from strain to strain. However, using the CLUSTAL alignment program (Baylor College of Medicine Nucleotide Search Launcher) to search for homologous sequences in the GenBank database, one of skill in the art can easily determine other SFB, and their corresponding genetic material. (Of course, other alignment programs may be used.) By way of non-limiting example, Bacillus subtilus (gb M10082) would be targeted at nucleotide 570 to nucleotide 930, SEQ ID NO. 52.

The polymerase chain reaction (PCR) is one technology which may be used to visualize the presence of sporulation genes. This method is based on the base complimentarily of DNA. DNA is composed of two anti-parallel strands composed of nucleotide “bases.” These bases, adenine, guanine, cytosine and thymine, form specific hydrogen bonds with one another. Adenine pairs with thymine and guanine pairs with cytosine. Strands of DNA can be denatured or converted to a single strand form by alkali or heat treatment. When conditions are favorable DNA will reassociate to its double stranded conformation.

The polymerase chain reaction (Mullis, U.S. Pat. Nos. 4,683,195, 4,683,202, and 4,800,159, the entire contents of each of which is incorporated by reference) is a commonly used method to amplify target DNA segments to detectable levels. It is currently being employed to detect many pathogenic bacteria. In this process, DNA primers of specific sequence, complementary to flanking regions of the target area, are used to prime enzymatic synthesis of DNA using a DNA polymerase. DNA polymerase requires a primer to initiate synthesis of a complementary DNA strand.

A number of different types of apparatuses and systems are available for performing PCR. Common apparatuses include Mini Cycler (MJ Instruments), Delta Cycler I System (EriComp), and Smart Cycler (Cepheid). Other systems may be used in accordance with the present invention as well. Examples are described in U.S. Pat. Nos. 5,882,496, 5,674,742, 5,646,039, 5,589,136, 5,639,423, each to NORTHRUP et al., 5,527,510, to ATWOOD et al., and 5,958,349, to PETERSEN et al. For their discussion of PCR systems, U.S. Pat. Nos. 5,882,496, 5,674,742, 5,646,039, 5,589,136, 5,639,423, each to NORTHRUP et al., 5,527,510, to ATWOOD et al., and 5,958,349, to PETERSEN et al. are incorporated herein by reference.

Primers are short (usually about 15-22 bases) stretches of nucleotides. Priming during PCR is controlled at the annealing step by temperature. Annealing conditions are experimentally determined for each primer set to allow for specificity. Following annealing, polymerization occurs as the polymerase synthesizes a complementary DNA strand. After polymerization, the PCR reaction is heated to denature all double stranded DNA. The use of a thermostable DNA polymerase, isolated from the hyperthermophile Thermus aquaticus, allows for repeated cycles of annealing, polymerization and denaturing to occur without loss of enzymatic activity. The process of PCR amplification is a routine laboratory process carried out in automated thermocyling units. The result is an exponential amplification of the targeted DNA segment. The amplified target may then be detected. The nucleotide sequences of the present invention, including SEQ ID NO. 1, SEQ ID NO. 2, SEQ ID NO. 3, SEQ ID NO. 4, SEQ ID NO. 5, SEQ ID NO. 6, SEQ ID NO. 7, and SEQ ID NO. 8., may be used in primers.

One method for detecting the presence of the amplified product is agarose gel electrophoresis, followed by staining. Other detection methods include, but are not limited to, fluorescence detection techniques. In one fluorescence-based technique, an intercalating dye such as Syber Green or ethidium bromide binds to double stranded DNA and then fluoresces. Incorporation of these dyes into PCR reactions result in an increase in fluorescence as the PCR reaction proceeds and double stranded DNA is synthesized. Thermal denaturation of the generated products can be used to ascertain the size and % GC (% GC is the number of G or C bases divided by the total number of bases) content of the PCR products generated.

In another technique, a tagged or labeled nucleotide sequence is used to detect hybridization. For example, a fluorescently tagged oligonucleotide sequence derived from an internal region of the spo0A PCR product can be used to detect the presence of the target in samples. As the PCR reaction proceeds the fluorescent tag is cleaved from the probe and fluorescence is observed. Increasing fluorescence is directly correlated with increased target in the test sample. Two examples of such sequences are 5′-AGTATCATTCATGAAATTGG-3′ (SEQ ID NO. 1) and 5′-AGTATCATTCATGAAATTGGCGTTCC-3′ (SEQ ID NO. 8). These sequences are presented for illustrative purposes; other stretches of conserved sequences within spo0A may also be targeted. Other nucleotide sequences of the present invention, including SEQ ID NO. 2, SEQ ID NO. 3, SEQ ID NO. 4, SEQ ID NO. 5, SEQ ID NO. 6, and SEQ ID NO. 7 would be useful in this regard.

Detection of hybridization between the nucleotide sequences of the present invention and the target may be achieved in a number of manners, in addition to those already mentioned. For example, it is envisioned that the nucleotide sequences of the present invention may be tagged or labled with a fluorescent or radioactive molecule. In the case of fluorescent labeling, the hybridized nucleotide sequence emits a different energy spectra than in non-hybridized form. With the radioactive probe, the hybridized sequence may be visualized by electrophoresing the hybridized sample, followed by exposure to a radiation-susceptible film. The nucleotide sequences of the present invention, including SEQ ID NO. 1, SEQ ID NO. 2, SEQ ID NO. 3, SEQ ID NO. 4, SEQ ID NO. 5, SEQ ID NO. 6, SEQ ID NO. 7, and SEQ ID NO. 8, are useful in this regard as probes.

The nucleotide sequences of the present invention were generated by sequence comparison of the spo0A gene from a broad spectrum of spore forming bacteria. This process entailed using a nucleic acid sequence alignment software program to elucidate highly conserved regions of the gene. From these regions, specific priming sites were chosen and appropriate primers were synthesized. Determination of the optimal sequences for primer selection is done by trial and error. Preferred primers meet all of the following criteria:

i. detection of spo0A from a characterized set of spore forming bacteria;

ii. negative results when testing non-SFB; and

iii. detection of spo0A from uncharacterized spore forming bacteria isolated from paper or paper manufacture samples.

It should be noted that the inventive nucleotide sequences disclosed herein are selected based on their ability to hybridize target genes of SFB. In particular, the instant nucleotide sequences are directed at conserved target genetic material of SFB. In considering which sequences will target the SFB, the instant nucleotide sequences of the present invention should be considered highly preferred. However, it is recognized that absolute identity to the sequences of the present invention may not be necessary to achieve a satisfactory result. That is, it is recognized that substitution of one or more bases may still allow hybridization to the target genes of SFB. Identity to the instant sequences is most preferred, and homologous or conservative substitutions are less preferred, but may still be acceptable. The trade-off will likely be a lower level of “inclusiveness,” that is, fewer species of SFB will be identified by the sequences in which substitutions have been made. In some applications, e.g., where identification of only one species of SFB is needed, this lower level of inclusiveness may be acceptable.

The methods of the present invention have been optimized to provide for detection of spore forming bacteria. In accordance with the present invention, spores may be detected at levels as low as 200 spores per gram of paper (and possibly even lower). The following steps allow for optimal detection:

a. 10 ml of 1% pulp sample (1 g pulp in 100 ml sterile water) is combined with 40 ml of tryptic soy broth medium (Difco Laboratories) and placed at 37° C. for 7 hours.

b. 4 ml of this sample are spun down to a pellet in a microcentrifuge tube.

c. The centrifuged pellet is washed in 100 μl sterile water (deionized) and centrifuged again.

d. The pellet is resuspended in 30 μl sterile water and boiled for 5 minutes.

e. 5 μl of the boiled solution is used for PCR and results are visualized on an agarose gel.

Note that a shorter incubation time in step a) may be used where there are higher concentrations of SFB. Also, some samples may require an even longer incubation period in step a). For example, a 16-hour incubation period may be used (but only 1 ml of the sample is centrifuged in step b)) for samples that are problematic. For example, longer incubation times may be used where very low numbers of SFB are believed present, or if a PCR amplification inhibitor is present. Thus, with a longer incubation period, detection to levels as low as approximately 100 spores/g paper may be achieved. Additionally, in step a), a process water, additive, or stock sample may be used instead of pulp as the starting sample. In step e), other visualization methods, e.g., fluorescence methods, may be used.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

The entire disclosures of all patents and publications, cited above and below, are hereby incorporated by reference as though set forth in full herein.

### EXAMPLES

### Example 1

Primer Set Number 1

A set of spo0A sequences is accessed through GenBank and aligned using the CLUSTAL alignment program. From the sequence alignment, oligonucleotide priming sites are selected and a preliminary primer set is chosen. A forward primer, 5′-AAAAAAGCAGTTGACT-3′ (SEQ ID NO. 2), and a reverse primer, 5′-CGGCTTGCCGTTGTATT-3′ (SEQ ID NO. 3), are synthesized. PCR products using this primer set are expected to be in the range of about 300 to 400 base pairs.—PCR reaction conditions are optimized using “Ready to Go”™ PCR beads (Pharmacia Biotech, Piscataway, N.J.) and different annealing temperatures for the thermocycling program. Any PCR apparatus may be used for this step, and the Mini Cycler and Delta Cycler are non-limiting examples thereof. Characterized SFB, as well as a set of uncharacterized SFB, isolated from a paper mill, are included in this test.

Uncharacterized samples (samples from a paper mill) testing positive are later tested and are shown to be positive for the presence of the spo0A gene. The results from the characterized SFB are shown in TABLE I.

TABLE I

| STRAIN | PCR PRODUCT? |
|---|---|
| Bacillus cereus | + |
| Bacillus subtilis | + |
| Bacillus megaterium | + |
| Clostridium perfringens | − |

The results show that all SFB except Clostridium perfringens showed a band with molecular weight size just under about 369 base pairs. To confirm that spo0A is being targeted, the agarose gel from the PCR is Southern blotted to nylon. As predicted, all SFB except Clostridium perfringens hybridized labeled amplification products from Bacillus cereus. However, even though Clostridium perfringens was not detected using this primer set, the positive results from uncharacterized paper mill samples indicate that this primer set is useful for its intended purpose.

### Example 2

Primer Set Number 2

Based on the results of Example 1, above, a second primer set is generated by comparison against a larger data set. TABLE II shows the data considered for the forward primer and TABLE III the data for the reverse primer of the refined primer set.

TABLE II

| | | | | | | | | | | | | |
|---|---|---|---|---|---|---|---|---|---|---|---|---|
| 9*-Bacillus cereus | | | | T | T | C | | | | | | |
| 10-Bacillus megaterium | | | | C | T | | C | | | | | |
| 11-Bacillus stearothermophilus | | | | C | T | | C | | | | | |
| 12-Bacillus thuringiensis | | | | T | T | | C | | | | | |
| 13-Bacillus sphaericus | | | | T | T | | T | | | | C | |
| 14-Bacillus anthracis | | | | T | T | | C | | | | | |
| 15-Clostridium pasteurianum | C | | | | T | T | C | T | C | | | |
| 16-Clostridium innocuum | T | C | T | C | T | C | T | | C | | T | |
| 17-Clostridium thermoaceticum | | | | T | T | | C | C | T | C | | T |

SEQ ID NOS

TABLE III

| | | | | | | | | | | | | | |
|---|---|---|---|---|---|---|---|---|---|---|---|---|---|
| 18*-Bacillus cereus | | T | C | | C | | | C | | | | C | C |
| 19-Bacillus megaterium | | T | C | | C | | | C | | | | C | C |
| 20-Bacillus stearothermophilus | | C | C | | C | | | C | | | | C | C |
| 21-Bacillus thuringiensis | | T | C | | C | | | C | | | | C | C |
| 22-Bacillus sphaericus | | T | C | | C | | C | C | | T | C | | C |
| 23-Bacillus anthacis | | T | C | | C | | | C | | | | C | C |
| 24-Clostridium pasteurianum | | T | C | T | C | T | | C | | | | C | C |
| 25-Clostridium innoculum | C | | C | C | C | | | C | | T | C | C | C |
| 26-Clostridium thermoaceticum | T | | C | T | C | T | C | C | C | | | T | C |

SEQ ID NOS
^{#}determined from plate counts
*broth culture of Bacillus cereus spores, no paper present

As can be seen from TABLES II and III, there is considerable sequence identity and homology in the genetic material of the SFB. Using the information from the sequence alignment, a new forward primer, 5′-GAAGATGTGACGAAAAAG-3′ (SEQ ID NO. 4) is synthesized. This primer, together with 5′-CGGCTTGCCGTTGTATT-3′ (SEQ ID NO. 3), described in Example 1 above, comprise the new primer set.

This primer set (SEQ ID NOS. 4 and 3) is tested individually against known SFB and non-SFB. This primer set yields spo0A products from characterized SFB and no products from non-SFB. Positive results are indicated by the presence of a band of 346-365 base pairs in size on an agarose gel, following PCR. TABLE IV shows the results from the characterized SFB which are tested.

TABLE IV

| STRAIN | PCR PRODUCT? |
|---|---|
| Bacillus cereus | + |
| Bacillus subtilis | + |
| Bacillus megaterium | + |
| Clostridium perfringens | + |
| Staphylococcus aureus | − |
| Staphylococcus epidermis | − |
| Pseudomonas aeruginosa | − |
| Kleibsiella pneumoniae | − |
| Bacillus stearothermophilus | + |
| Bacillus lichenformis | + |

As can be seen from TABLE IV, Clostridium perfringens, in addition to the other species, is detected using the new primer set. As can be seen from the results, this primer set exhibited the desired characteristics: hybridization to SFB, and no hybridization to non-SFB. Also, as with the previous primer set (from Example 1), positive results from uncharacterized paper mill samples confirms that this primer set works for its intended purpose.

Once it is determined that the primer set did perform its intended function, additional tests are performed to determine how sensitive the primer set is. The following procedure is therefore performed to determine the “detection limits” for the primer set. Although this procedure used paper samples as test materials, the procedure is adaptable to testing all manners of samples, including air, soil, food, and water, including but not limited to, waste water, industrial process water, and drinking water. It should be noted that in Example 3, below, this procedure is further refined and optimized.

Determining Detection Limits—

1. A 100 ml culture of Bacillus cereus is grown to lag phase and then placed at 80° C. to induce sporulation.

2. This culture is diluted in 10-fold increments in phosphate buffered saline and 0.1 ml of the dilutions are spotted onto 0.5 g paper samples of different types and grades including A) Kraft liner board, recycled, B) alkaline kraft paper, and C) acid fine paper.

3. Paper samples are then placed in 10 ml phosphate buffered saline (PBS) and vortexed for 2 minutes.

4. The samples are then placed at 80° C. for 10 minutes and 1 (one) ml of the sample is placed into 9 ml PBS to obtain another 10-fold dilution.

5. 0.1 ml of the sample is added to a sterile microfuge tube containing 0.1 ml tryptic soy medium (0.1 ml of the sample and sample dilution are plated to correlate PCR result with colony forming units).

6. The samples are incubated at 37° C. for 45 minutes to allow for germination. [Note: this step is optimized in Example 3. A longer incubation time may be necessary for lower bacterial concentrations.]

7. The microfuge tubes are boiled for 5 minutes and 5 μl are used for PCR using “Ready to Go”™ PCR beads (Pharmacia).

8. The thermocycler program is set as follows:

a. 5 minutes at 94° C.

b. 30 cycles of: 0.5 minutes at 94° C., 0.5 minutes at 52° C., 0.5 minutes at 72° C.

c. 3 minutes at 72° C.

The detection limits established for this primer set are set forth in TABLE V.

TABLE V

| Sample | Spores/0.5 g Paper^{#} | spo0A PCR Product? |
|---|---|---|
| no paper* | 171 ± 6.0 | + |
| no paper | 22 ± 1.0 | − |
| A | 114.5 ± 1.5 | + |
| A | 1.5 ± 0.5 | − |
| B | 59 ± 6.0 | + |
| B | 7 ± 7.0 | − |
| C | 149 ± 1.0 | + |
| C | 19.5 ± 18.5 | − |

^{#}determined from plate counts
*broth culture of Bacillus cereus spores, no paper present

In TABLE V, A is kraft liner board, recycled, B is alkaline kraft paper, and C is acid fine paper.

### Example 3

Primer Set Number 3

The primer set from Example 2 may inconsistently detect Bacillus sphaericus. In order to address this problem, a new primer set is prepared. The data considered in preparing the refined primer set is shown in TABLE VI (forward primer) and in TABLE VII (reverse primers).

TABLE VI

| | | | | | | | | | | | | | |
|---|---|---|---|---|---|---|---|---|---|---|---|---|---|
| 27*-Bacillus cereus | C | | | | | T | T | | C | | | | A |
| 28-Bacillus megaterium | C | | | | | C | T | | C | | | | A |
| 29-Bacillus stearothermophilus | C | | | | | C | T | | C | | | | A |
| 30-Bacillus thuringiensis | C | | | | | T | T | | C | | | | A |
| 31-Bacillus sphaericus | C | | | | | T | T | | T | | | | A |
| 32-Bacillus anthracis | C | | | | | T | T | | C | | | | A |
| 33-Bacillus subtilis | C | | | | | T | T | C | C | | | | A |
| 34-Clostridium pasteurianum | C | | C | | | | T | T | C | C | | | A |
| 35-Clostridium innocuum | | C | T | C | T | C | T | C | T | | | C | A |
| 36-Clostridium thermoaceticum | C | | | | | T | T | | C | C | | T | G |
| 37-Clostridium perfringens | C | | C | | | | T | T | C | T | C | A | |

SEQ ID NOS.

TABLE VII

| | | | | | | | | | | | | | | |
|---|---|---|---|---|---|---|---|---|---|---|---|---|---|---|
| 38*-Bacillus cereus | C | | | | | T | | T | | T | C | A | A | C |
| 39-Bacillus megaterium | | | | | | T | | T | | T | C | A | A | C |
| 40-Bacillus stearothermophilus | C | C | | | | T | | C | | C | C | A | A | C |
| 41-Bacillus thuringiensis | C | | | | | T | | T | | T | C | A | A | C |
| 42-Bacillus sphaericus | C | | | | | T | T | C | | T | C | A | A | C |
| 43-Bacillus anthracis | C | | | | | T | | T | | T | C | A | A | C |
| 44-Macillus subtilis | C | C | | | | T | T | T | | C | C | A | A | C |
| 45-Clostridium pasteurianum | C | | | | | T | | T | | T | C | T | A | C |
| 46-Clostridium innoculum | C | C | | | | T | | T | C | | C | C | A | C |
| 47-Clostridium thermoaceticum | C | C | C | C | | T | | T | T | | C | T | A | C |
| 48-Clostridium perfringens | C | | | | C | T | | C | | | C | T | T | T |

SEQ ID NOS.

Using the information from the sequence alignment, a new primer set is prepared. The new set comprises one forward primer and two reverse primers. The new set is: 5′-CAAGAAGATGTGACGAAA-3′ (SEQ ID NO. 5) (forward), 5′-GTTGTATTATATTTCTTTGC-3′ (SEQ ID NO. 6) (reverse), and 5′-GTTGTGTTAAATTTTTTGGC-3′ (SEQ ID NO. 7) (reverse).

This primer set yields spo0A products from characterized SFB and no products from non-SFB. Positive results are indicated by the presence of a band of 347-356 base pairs in size on an agarose gel, following PCR. TABLE VII shows the results from the characterized SFB which are tested.

TABLE VIII

| STRAIN | PCR PRODUCT? |
|---|---|
| Bacillus cereus | + |
| Bacillus subtilis (ATCC 6051) | + |
| Bacillus subtilis (ATCC 23059) | + |
| Bacillus megaterium | + |
| Bacillus stearothermophilus | + |
| Bacillus lichenformis | + |
| Bacillus sphaericus | + |
| Clostridium perfringens | + |
| Staphylococcus aureus* | − |
| Staphylococcus epidermis* | − |
| Staphylococcus pyogenes* | − |
| Pseudomonas aeruginosa* | − |
| Kleibsiella pneumoniae* | − |

*Non-SFB

Once it is determined that the new primer set performed as intended, tests are preformed to determine the limits of detection. The procedure for determining the limits of detection is similar to that in Example 2 above, with some exceptions.

1. 10 ml of 1% pulp sample (food-grade packaging board) is combined with 40 ml tryptic soy broth medium and placed at 37° C. for 7 hours.

2. 4 ml of sample are centrifuged to a pellet in a microcentrifuge tube.

3. The pellet is washed in 100 μl sterile water and centrifuged again.

4. The pellet is resuspended in 30 μl sterile water and boiled for 5 minutes.

5. 5 μl of the boiled solution is used in the polymerase chain reaction.

As noted above, some samples may require incubation time as long as 16 hours to optimize detection. (Longer periods are believed to be needed when the sample has a high concentration of clay, or other contaminant.) When a 16-hour incubation is employed, only 1 ml of sample is pelleted. The longer period may improve detection to as low as 100 spores/g paper.

The detection limits determined for the Example 3 primer set are shown in TABLE IX below.

TABLE IX

| Sample | Spores/0.5 g Paper^{#} | spo0A PCR Product? |
|---|---|---|
| A | 605 ± 43 | + |
| B | 590 ± 90 | + |
| C | 520 ± 50 | + |
| D | 340 ± 0 | + |
| E | 255 ± 55 | − |
| F | 175 ± 15 | − |

^{#}determined from plate counts

In TABLE IX, samples A-F are all food-grade packaging board samples of the same type, spiked with different levels of SFB.

### Example 4

Detecting Spore Forming Bacteria in Paper Making Process

Samples of 10 ml are taken from process water in the head box area of the paper mill. The samples are separately mixed with 40 ml tryptic soy broth medium. Following a 7-hour incubation period, samples are centrifuged to concentrate bacterial contents. The supernatant is decanted and the pellet resuspended.

The resuspended sample is boiled to lyse the bacteria, and the lysed sample cooled and mixed with primers prior to placing the test mixture in a PCR thermocycler. The thermocycler is run and the PCR results are electrophoresed on an agarose gel, stained with ethydium bromide, and visualized under an ultraviolet light.

If spore forming bacterial counts are shown to be unacceptably high, biocide is added to kill the bacteria.

### Example 5

Detecting Spore Forming Bacteria in a Food Making Process

Samples of 10 ml are taken from milk being processed both prior to, and after, pasteurization. Samples are also periodically checked in the packaged product as well. Each 10 ml sample to be tested is separately mixed with 40 ml tryptic soy broth medium. Following a 7-hour incubation period, samples are centrifuged to concentrate bacterial contents. The supernatant is decanted and the pellet resuspended.

The resuspended sample is boiled to lyse the bacteria, and the lysed sample cooled and mixed with primers prior to placing the test mixture in a PCR thermocycler. The thermocycler is run and the PCR results are electrophoresed on an agarose gel, stained with ethydium bromide, and visualized under an ultraviolet light.

Based upon the results of the testing, appropriate measures may be taken to eradicate the spore forming bacteria at the appropriate stage in the process.

### Example 6

Detecting Spore Forming Bacteria in a Biological Sample

Samples of 100 mg are taken from fecal matter to be tested. Each 100 mg sample to be tested is separately mixed with 50 ml tryptic soy broth medium. Following a 7-hour incubation period, samples are centrifuged to concentrate bacterial contents. The supernatant is decanted and the pellet resuspended.

The resuspended sample is boiled to lyse the bacteria, and the lysed sample cooled and mixed with primers prior to placing the test mixture in a PCR thermocycler. The thermocycler is run and the PCR results are electrophoresed on an agarose gel, stained with ethydium bromide, and visualized under an ultraviolet light.

Based upon the results of the testing, an antibiotic which is effective at treating a spore forming bacterial infection is prescribed.

In each of Examples 4, 5, and 6 (and in other embodiments as well), contaminants may interfere with the ability of the test method to detect spore forming bacteria. For example, the presence of clays, or some enzymes, in a sample may result in an interference with polymerase chain reaction. In such cases, it is recommended that dilution of the original sample be performed until the contaminants are no longer present at an interfering concentration.

Also, with regard to each of Examples 4, 5, and 6, it is noted that while PCR is taught as a method for detecting hybridization of the probes to the target sample, other methods may be used. For example, a probe may be linked to a fluorescent (other other detectable) molecule prior to mixing with the sample. Upon hybridization, and under the proper conditions, the tagged molecule will give off a detectable energy, e.g., fluorescence.

From the foregoing descriptions, one skilled in the art can easily ascertain the essential characteristics of this invention, and without departing from the spirit and scope thereof, can make various changes and modifications of the invention to adapt it to various usages and conditions.

For example, the inventive nucleotide sequences disclosed herein are selected based on their ability to hybridize target genes of SFB. In particular, the instant nucleotide sequences are directed at conserved target genetic material of SFB. Thus, it is believed that other nucleotide sequences which bind to the target area of SFB genes are within the scope of the present invention.

However, it is recognized that substitution of bases within the inventive nucleotide sequences may still result in hybridization to the target genes. Such substitutions are believed to be within the scope of the present invention, and should amount to an insubstantial difference therefrom.

Additionally, as has been shown, the inventive nucleotide sequences can be combined with other nucleotide sequences and still achieve the same result. This effect is demonstrated in Examples 1 and 2, where modifying only one of the two primers resulted in improved detection. Thus, it is believed that the combinations of the present inventive nucleotide sequences with other nucleotide sequences is within the scope of the present invention.

What is claimed is:

1. A primer pair comprising at least one member selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, and SEQ ID NO. 8.

2. The primer pair of claim 1, wherein the at least one member is selected from the group consisting of SEQ ID NO. 5, SEQ ID NO. 6, and SEQ ID NO. 7.

3. A primer selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, and SEQ ID NO. 8.

4. The primer of claim 3 wherein the primer is selected from the group consisting of SEQ ID NO. 5, SEQ ID NO. 6, and SEQ ID NO. 7.

5. A composition comprising at least one primer comprising a sequence selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, and SEQ ID NO. 8.

6. The composition of claim 5 further comprising at least one cellulose-containing material.

7. The composition of claim 6, wherein the at least one cellulose-containing material comprises paper pulp.

8. The composition of claim 6 wherein the at least one primer comprising a sequence comprises a sequence selected from the group consisting of SEQ ID NO. 5, SEQ ID NO. 6, and SEQ ID NO. 7.

9. The composition of claim 8, wherein the cellulose-containing material comprises paper pulp.

10. A kit for testing for the presence of spore forming bacteria in a paper-making process comprising:
a) a primer comprising a sequence selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, and SEQ ID NO. 8, and
b) at least one growth medium supportive of spore forming bacterial growth.

11. The kit of claim 10 wherein the primer comprising the sequence comprises a sequence selected from the group consisting of SEQ ID NO. 5, SEQ ID NO. 6, and SEQ ID NO. 7.

12. A probe for detecting the presence of spore forming bacteria in a sample, the probe comprising a nucleotide sequence able to form a detectable hybrid with spo0A gene of spore forming bacteria and unable to form a detectable hybrid with genetic material of non-spore forming bacteria, wherein the nucleotide sequence is able to form a detectable hybrid to bases 70 to 427 of the spo0A gene of Bacillus cereus, corresponding to GenBank accession number gb U09972, SEQ. ID NO. 51, or in the alternative to form a detectable hybrid to bases 403 to 422 of the spo0A gene of Bacillus cereus, corresponding to GenBank accession number gb U09972, SEQ. ID NO. 50.

13. The probe of claim 12 wherein the nucleotide sequence is able to form a detectable hybrid to bases 403 to 422 and wherein the nucleotide sequence comprises at least one of SEQ. ID. NO. 6 and SEQ ID NO. 7.

14. The probe of claim 12 wherein the nucleotide sequence is able to form a detectable hybrid to bases 70 to 427 and wherein the nucleotide sequence comprises at least one of SEQ ID NO. 1, SEQ ID NO. 2, SEQ ID NO. 3, SEQ ID NO. 4, SEQ ID NO. 5, SEQ ID NO. 6, SEQ ID NO. 7, and SEQ ID NO. 8.

15. A probe for detecting the presence of spore forming bacteria in a sample, the probe comprising a nucleotide sequence able to form a detectable hybrid with spo0A gene of spore forming bacteria and unable to form a detectable hybrid with genetic material of non-spore forming bacteria, wherein the nucleotide sequence is able to form a detectable hybrid to bases 76 to 93 of the spo0A gene of Bacillus cereus, corresponding to GenBank accession number gb U09972, SEQ ID NO. 49.

16. The probe of claim 15, wherein the nucleotide sequence comprises SEQ ID NO. 5.

17. The probe of claim 12, wherein the nucleotide sequence is able to form a detectable hybrid to bases 70 to 427 of the spo0A gene of Bacillus cereus, corresponding to GenBank accession number gb U09972, SEQ. ID NO. 51, and the nucleotide sequence consisting essentially of guanine, cytosine, adenine, and thymine.

18. A probe for detecting the presence of spore forming bacteria in a sample comprising a nucleic acid sequence able to form a detectable hybrid with a sufficient number of highly conserved regions of the spo0A gene of spore forming bacteria Bacillus and Clostridium bacteria species set out in either (a) of (b) below
a) Bacillus cereus
Bacillus megaterium
Bacillus subtilis, and
Clostridium pasteurianum;
b) Bacillus cereus
Bacillus megaterium
Bacillus sphaericus, and
Clostridium pasteurianum
said nucleic acid sequence being unable to form a detectable hybrid with genetic material of non-spore forming bacteria.

19. The probe of claim 18, wherein the nucleic acid sequence comprises at least one of SEQ ID NO. 1, SEQ ID NO. 2, SEQ ID NO. 3, SEQ ID NO. 4, SEQ ID NO. 5, SEQ ID NO. 6, SEQ ID NO. 7, and SEQ ID NO. 8.

20. The probe of claim 18, wherein the nucleic acid sequence acts as a primer in a polymerase chain reaction used to amplify the detectable hybrid.

21. The probe of claim 18, wherein the nucleic acid sequence is tagged with a fluorescent derivative for detection of the detectable hybrid.

22. The probe of claim 18, wherein the nucleic acid is tagged with a radioisotope for detection of the detectable hybrid.


SEQUENCE LISTING (52 sequences)

SEQ ID NO: 1 - DNA, 20 bp, Bacillus cereus
agtatcattcatgaaattgg

SEQ ID NO: 2 - DNA, 16 bp, Bacillus cereus
aaaaaagcagttgact

SEQ ID NO: 3 - DNA, 17 bp, Bacillus cereus
cggcttgccgttgtatt

SEQ ID NO: 4 - DNA, 18 bp, Bacillus cereus
gaagatgtgacgaaaaag

SEQ ID NO: 5 - DNA, 18 bp, Bacillus cereus
caagaagatgtgacgaaa

SEQ ID NO: 6 - DNA, 20 bp, Bacillus cereus
gttgtattatatttctttgc

SEQ ID NO: 7 - DNA, 20 bp, Bacillus cereus
gttgtgttaaattttttggc

SEQ ID NO: 8 - DNA, 26 bp, Bacillus cereus
agtatcattcatgaaattggcgttcc

SEQ ID NO: 9 - DNA, 19 bp, Bacillus cereus
gaagatgtgacgaaaaaag

SEQ ID NO: 10 - DNA, 19 bp, Bacillus megaterium
gaagacgtaacgaaaaaag

SEQ ID NO: 11 - DNA, 19 bp, Bacillus stearothermophilus
gaagacgtgacgaaaaagg

SEQ ID NO: 12 - DNA, 19 bp, Bacillus thuringiensis
gaagatgtgacgaaaaaag

SEQ ID NO: 13 - DNA, 19 bp, Bacillus sphaericus
gaagatgtaatgaaacagg

SEQ ID NO: 14 - DNA, 19 bp, Bacillus anthracis
gaagatgtgacgaaaaaag

SEQ ID NO: 15 - DNA, 19 bp, Clostridium pasteurianum
gacaaaattactcaaagag

SEQ ID NO: 16 - DNA, 19 bp, Clostridium innocuum
gatctcatcgtggcaggtg

SEQ ID NO: 17 - DNA, 19 bp, clostridium thermoaceticum
gagagtatgaccatgcggt

SEQ ID NO: 18 - DNA, 17 bp, Bacillus cereus
aatacaacagcaagccg

SEQ ID NO: 19 - DNA, 17 bp, Bacillus megaterium
aatacaacggcaagccg

SEQ ID NO: 20 - DNA, 17 bp, Bacillus stearothermophilus
aacacaacggcaagccg

SEQ ID NO: 21 - DNA, 17 bp, Bacillus thuringiensis
aatacaacagcaagccg

SEQ ID NO: 22 - DNA, 17 bp, Bacillus sphaericus
aatacaacaccgtcacg

SEQ ID NO: 23 - DNA, 17 bp, Bacillus anthracis
aatacaacagcaagccg

SEQ ID NO: 24 - DNA, 17 bp, Clostridium pasteurianum
aatactactgcaagccg

SEQ ID NO: 25 - DNA, 17 bp, Clostridium innocuum
gcaaccacggcatcccg

SEQ ID NO: 26 - DNA, 17 bp, clostridium thermoaceticum
atgactactcccagtcg

SEQ ID NO: 27 - DNA, 18 bp, Bacillus cereus
caagaagatgtgacgaaa

SEQ ID NO: 28 - DNA, 18 bp, Bacillus megaterium
caagaagacgtaacgaaa

SEQ ID NO: 29 - DNA, 18 bp, Bacillus stearothermophilus
caggaagacgtgacgaaa

SEQ ID NO: 30 - DNA, 18 bp, Bacillus thuringiensis
caagaagatgtgacgaaa

SEQ ID NO: 31 - DNA, 18 bp, Bacillus sphaericus
caagaagatgtaatgaaa

SEQ ID NO: 32 - DNA, 18 bp, Bacillus anthracis
caagaagatgtgacgaaa

SEQ ID NO: 33 - DNA, 18 bp, Bacillus subtilis
caggaagatgtcacgaaa

SEQ ID NO: 34 - DNA, 18 bp, Clostridium pasteurianum
caagacaaaattaccaaa

SEQ ID NO: 35 - DNA, 18 bp, Clostridium innocuum
aacgatctcatcgtggca

SEQ ID NO: 36 - DNA, 18 bp, clostridium thermoaceticum
caggagagtatgaccatg

SEQ ID NO: 37 - DNA, 18 bp, Clostridium perfringens
caagacaaaattactcaa

SEQ ID NO: 38 - DNA, 20 bp, Bacillus cereus
gcaaagaaatataatacaac

SEQ ID NO: 39 - DNA, 20 bp, Bacillus megaterium
gaaaaaaaatataatacaac

SEQ ID NO: 40 - DNA, 20 bp, Bacillus stearothermophilus
gccaaaaaatacaacacaac

SEQ ID NO: 41 - DNA, 20 bp, Bacillus thuringiensis
gcaaagaaatataatacaac

SEQ ID NO: 42 - DNA, 20 bp, Bacillus sphaericus
gcaaagaaattcaatacaac

SEQ ID NO: 43 - DNA, 20 bp, Bacillus anthracis
gcgaagaaatataatacaac

SEQ ID NO: 44 - DNA, 20 bp, Bacillus subtilis
gccaaaaaatttaacacaac

SEQ ID NO: 45 - DNA, 20 bp, Clostridium pasteurianum
gcaaaaaaatataatactac

SEQ ID NO: 46 - DNA, 20 bp, Clostridium innocuum
gccaagaaatatgcaaccac

SEQ ID NO: 47 - DNA, 20 bp, clostridium thermoaceticum
gcccgcaagtatatgactac

SEQ ID NO: 48 - DNA, 20 bp, Clostridium perfringens
  misc_feature (3)..(4): a or c or g or t
gcnnaggcatgcaaggcttt

SEQ ID NO: 49 - DNA, 18 bp, Bacillus cereus
caagaagatgtgacgaaa

SEQ ID NO: 50 - DNA, 20 bp, Bacillus cereus
gcaaagaaatataatacaac

SEQ ID NO: 51 - DNA, 357 bp, Bacillus cereus
ttggccaagaagatgtgacgaaaaaagcagttgacttaggtgcctcgtatttcatattaaaaccattcgatatggagaatttaccgagtcatattcgtcaagtgagtggtaaagcaaacactatgattaagcgtccactaccatcattccgatcagccacaacagtagatggaaaaccgaaaaacttagatgcgagtattacgagtatcattcatgaaattggtgtacccgctcatattaaaggatatatgtatttacgagaagcaatctccatggtatacaatgatatcgaattattaggatcgattacgaaagtattgtatccagatatcgcaaagaaatataatacaacagcaa

SEQ ID NO: 52 - DNA, 370 bp, Bacillus subtilis
ctgacagcctttgggcaggaagatgtcacgaaaaaggccgtcgatttaggcgcgtcctactttattctcaaaccgtttgatatggaaaaccttgtcggccatatccgccaggtcagcggaaatgccagcagtgtgacgcatcgtgcgccatcatcgcaaagcagtattatacgcagcagccagcctgaaccaaagaagaaaaatctcgacgcgagcatcacaagcattatccatgaaatcggcgtcccagcccatattaaaggctatctctatctgcgcgaagcaatctcaatggtatacaatgacatcgaattgctcggcagcattacaaaagtcctctatccggacatcgccaaaaaatttaacacaa